(19) United States
(12) Patent Application Publication
(10) Pub. No.: US 2011/0002953 A1
(43) Pub. Date: Jan. 6, 2011

(54) USE OF IMMIDAZOQUINOLINAMINES AS ADJUVANTS IN DNA VACCINATION

(76) Inventors: Lindy Louise THOMSEN, Stevenage (GB); John Philip Tite, Stevenage (GB); Peter Topley, Stevenage (GB)

Correspondence Address: GLAXOSMITHKLINE;Corporate Intellectual Property - UW2220, P.O.Box 1539, King of Prussia, PA, 19406-0939, US

(21) Appl. No.: 12/882,470

(22) Filed: Sep. 15, 2010

Related U.S. Application Data
(63) Continuation of application No. 11/764,814, filed on Jun. 19, 2007.
Application No. 11/764,814 is a continuation of application No. 10/380,981, filed on Sep. 23, 2003, now abandoned.

(30) Foreign Application Priority Data
Sep. 20, 2000 (GB) 0023008.6

(51) Int. Cl. A61K 39/00

(52) U.S. Cl. 424/184.1

(57) ABSTRACT

The present invention relates to the use of a 1H-imidazo[4,5-c]quinolin-4-amine derivative as an adjuvant for use with nucleic acid vaccination.

### CROSS REFERENCE TO PRIOR APPLICATION

This is a continuation of application Ser. No. 11/764,814, filed 19 Jun. 2007, which is a continuation of application Ser. No. 10/380,981, filed 23 Sep. 2003, which is a 371 application of PCT/GB01/04207 filed 20 Sep. 2001.

### FIELD OF THE INVENTION

The present invention relates to improvements in DNA vaccination and in particular, but not exclusively, to vaccine compositions, methods of vaccinating a mammal against disease states, and to the use of certain compounds in the manufacture of medicaments.

### BACKGROUND OF THE INVENTION

Traditional vaccination techniques which involve the introduction into an animal system of an antigen which can induce an immune response in the animal, and thereby protect the animal against infection, have been known for many years. Following the observation in the early 1990's that plasmid DNA could directly transfect animal cells in vivo, significant research efforts have been undertaken to develop vaccination techniques based upon the use of DNA plasmids to induce immune responses, by direct introduction into animals of DNA which encodes for antigenic peptides. Such techniques, which are referred to as “DNA immunisation” or “DNA vaccination” have now been used to elicit protective antibody (humoral) and cell-mediated (cellular) immune responses in a wide variety of pre-clinical models for viral, bacterial and parasitic diseases. Research is also underway in relation to the use of DNA vaccination techniques in treatment and protection against cancer, allergies and autoimmune diseases.

DNA vaccines usually consist of a bacterial plasmid vector into which is inserted a strong promoter, the gene of interest which encodes for an antigenic peptide and a polyadenylation/transcriptional termination sequence. The immunogen which the gene of interest encodes may be a full protein or simply an antigenic peptide sequence relating to the pathogen, tumour or other agent which is intended to be protected against. The plasmid can be grown in bacteria, such as for example E. coli and then isolated and prepared in an appropriate medium, depending upon the intended route of administration, before being administered to the host.

Helpful background information in relation to DNA vaccination is provided in “Donnelly, J et al Annual Rev. Immunol. (1997) 15:617-648, the disclosure of which is included herein in its entirety by way of reference.

There are a number of advantages of DNA vaccination relative to traditional vaccination techniques. First, it is predicted that because the proteins which are encoded by the DNA sequence are synthesised in the host, the structure or conformation of the protein will be similar to the native protein associated with the disease state. It is also likely that DNA vaccination will offer protection against different strains of a virus, by generating cytotoxic T lymphocyte responses that recognise epitopes from conserved proteins. Furthermore, because the plasmids are introduced directly to host cells where antigenic protein can be produced, a long-lasting immune response will be elicited. The technology also offers the possibility of combining diverse immunogens into a single preparation to facilitate simultaneous immunisation in relation to a number of disease states.

Despite the numerous advantages associated with DNA vaccination relative to traditional vaccination therapies, there is nonetheless a desire to develop adjuvant compounds which will serve to increase the immune response induced by the protein which is encoded by the plasmid DNA administered to an animal.

DNA vaccination is sometimes associated with an inappropriate deviation of immune response from a Th1 to a Th2 response, especially when the DNA is administered directly to the epidermis (Fuller and Haynes Hum. Retrovir. (1994) 10:1433-41). It is recognised that the immune profile desired from a nucleic acid vaccine depends on the disease being targeted. The preferential stimulation of a Th1 response is likely to provide efficacy of vaccines for many viral diseases and cancers, and a dominant Th2 type of response may be effective in limiting allergy and inflammation associated with some autoimmune diseases. Accordingly, ways to quantitatively raise the immune response or to shift the type of response to that which would be most efficacious for the disease indication, may be useful.

Accordingly, it is one object of the present invention to provide adjuvant compounds which can be used in conjunction with DNA vaccination procedures. It is also an object to provide compositions including the adjuvants concerned, as well as methods of improved DNA vaccination involving such adjuvants. Other objects of the present invention will become apparent from the following detailed description thereof. To date, however, meeting these objects has proven difficult, largely due to mechanistic differences associated with DNA vaccination, as compared with traditional vaccine techniques. Also, identification of suitable compounds is not straightforward, a number of known immunopotentiating agents have been tried in combination with DNA vaccination techniques with limited, or at best mixed success. For example, co-administration of the genes for IFN-γ and rabies virus glycoprotein had an inhibitory effect on both T helper cell response and the antibody response (Xiang and Ertl).

With this background in mind, it is most surprising to note that the present inventors report adjuvant compounds that are effective in promoting an improved immune response, in particular an improved cellular immune response when used as adjuvants in DNA vaccination. Imidazoquinolineamine derivatives are inducers of cytokines, including IFN-α, IL-6 and TNF-α (See, e.g. Reiter et al, J. Leukocyte Biology (1994) 55:234-240). These compounds and processes for their preparation have been disclosed in PCT patent application publication number WO 94/17043. The present inventors have shown that these derivatives may be effectively used as adjuvants in DNA vaccination.

### SUMMARY OF THE INVENTION

According to one embodiment of the present invention there is provided a vaccine composition comprising (i) an adjuvant component comprising a 1H-imidazo[4,5-c]quinolin-4-amine derivative and (ii) an immunogen component comprising a nucleotide sequence encoding an antigenic peptide or protein associated with a disease state, wherein the adjuvant component enhances the immune responses in a mammal to the antigenic peptide or protein.

In a further aspect, the invention provides a method of increasing an immune response to an antigen, said method comprising administration, either sequentially or simultaneously, a nucleic acid encoding an antigen and an imidazo[4,5-c]quinolin-4-amine derivative.

In a further embodiment there is provide the use of an imidazo[4,5-c]quinolin-4-amine derivative in the manufacture of a medicament for the enhancement of an immune response to an antigen encoded by a nucleotide sequence, said nucleotide sequence being administered either sequentially or simultaneously with said derivative.

In a further embodiment the present invention further provides a pharmaceutical composition comprising an imidazo[4,5-c]quinolin-4-amine derivative to enhance an immune response to an antigen encoded by a nucleic acid sequence.

Preferably the 1H-imidazo[4,5-c]quinolin-4-amine-derivative is a compound defined by one of formulae I-VI defined herein. More preferably, it is a compound defined by formula VI. Particularly preferred is when the 1H-imidazo[4,5-c]quinolin-4-amine derivative is a compound of formula VI selected from the group consisting of
1-(2-methylpropyI)-1H-imidazo[4,5-c]quinolin-4-amine;
1-(2-hydroxy-2-methylpropyl)-2-methyl-1H-imidazo[4,5-c]quinolin-4-amine;
1-(2hydroxy-2-methylpropyl)-1H-imidazo[4,5-c]quinolin-4-amine;
1-(2-hydroxy-2-methylpropyl)-2-ethoxymethyl-1-H-imidazo[4,5-c]quinolin-4-amine

In another embodiment, the present invention provides a method of raising an immune response in a mammal against a disease state, comprising administering to said mammal within an appropriate vector, a nucleotide sequence encoding an antigenic peptide associated with the disease state; additionally administering to said mammal as a vaccine adjuvant a 1H-imidazo[4,5-c]quinolin-4-amine derivative to raise said immune response. Further provided is a method of increasing the immune response of a mammal to an immunogen, comprising the step of administering to said mammal, within an appropriate vector, a nucleotide sequence encoding said immunogen in an amount effective to stimulate an immune response; additionally administering to said mammal as a vaccine adjuvant a 1H-imidazo[4,5-c]quinolin-4-amine derivative in an amount effective to increase said immune response. Preferably in said methods, the 1H-imidazo[4,5-c]quinolin-4-amine derivative is a compound of formula VI.

In a further embodiment, the present invention provides the use of a 1H-imidazo[4,5-c]quinolin-4-amine derivative in the manufacture of a medicament for enhancing immune responses initiated by an antigenic peptide or protein, said peptide/protein being expressed as a result of administration to a mammal of a nucleotide sequence encoding for said peptide.

### BRIEF DESCRIPTION OF THE FIGURES

FIG. 1.

Imiquimod increases the cytotoxic T-cell response following vaccination with a plasmid encoding nucleoprotein from influenza virus (pVAC1.PR). pVAC1 is the vector control.

FIG. 2.

Imiquimod increases the clonal expansion of CD4 T cells in vivo following vaccination with a plasmid encoding ovalbumin protein, as measured by increased proliferation of CD4+ T cells.

FIG. 3.

Imiquimod increases the number of activated CD4 T cells in vivo following vaccination with a plasmid encoding ovalbumin protein, as measured by increased IFN-γ and IL-4 producing cells.

FIG. 4.

Imiquimod induces both Th1 and Th2 responses in vivo following vaccination with a plasmid encoding ovalbumin protein, as measured by increased IFN-γ and IL-4 producing cells, respectively.

FIG. 5.

Imiquimod increases the cytotoxic T-cell response following vaccination with a plasmid encoding the HIV antigens Gag and Nef (WRG7077.Gag/Nef). WRG7077 is the vector control.

FIG. 6.

Resiquimod (1-(2-hydroxy-2-methylpropyl)-2-ethoxymethyl-1-H-imidazo[4,5-c]quinolin-4-amine), an analogue of imiqumod, increases the number of activated CD4 T cells in vivo following vaccination with a plasmid encoding ovalbumin protein, as measured by increased IFN-γ and IL-4 producing cells (6A and 6B, respectively).

FIG. 7.

Two analogues of imiquimod, 1-(2-hydroxy-2-methylpropyl)-2-methyl-1H-imidazo[4,5-c]quinolin-4-amine and 1-(2,hydroxy-2-methylpropyl)-1H-imidazo[4,5-c]quinolin-4-amine, increase the number of activated CD4 T cells in vivo following vaccination with a plasmid encoding ovalbumin protein, as measured by increased IFN-γ and IL-4 producing cells (7A and 7B, respectively).

FIG. 8.

Topical application of imiquimod increases the cytotoxic T-cell response following vaccination with a plasmid encoding the HIV antigens Gag and Nef (WRG7077.Gag/Nef). WRG7077 is the vector control.

FIG. 9.

Imiquimod delays the growth of tumours in animals challenged with ovalbumin-expressing EG7.OVA tumour cells after immunisation with the plasmid encoding ovalbumin.

FIG. 10.

Imiquimod reduces the tumourigenicity of ovalbumin-expressing EG7.OVA tumour cells implanted into animals pre-immunised with the plasmid encoding ovalbumin.

### DETAILED DESCRIPTION OF THE INVENTION

Throughout this specification and the appended claims, unless the context requires otherwise, the words “comprise” and “include” or variations such as “comprising”, “comprises”, “including”, “includes”, etc., are to be construed inclusively, that is, use of these words will imply the possible inclusion of integers or elements not specifically recited.

As described above, the present invention relates to immunogenic compositions such as vaccine compositions, vaccination methods, and to improvements of methods of vaccination involving the introduction into a mammal of nucleotide sequence which encodes for an immunogen which is an antigenic protein or peptide, such that the protein or peptide will be expressed within the mammalian body to thereby induce an immune response within the mammal against the antigenic protein or peptide. Such methods of vaccination are well known and are fully described in Donnelly et al as referred to above.

As used herein the term vaccine composition refers to a combination of a immunogen component comprising a nucleotide sequence encoding an immunogen, and an adjuvant component comprising a 1H-imidazo[4,5-c]quinolin-4-amine derivative. The combination is, for example, in the form of an admixture of the two components in a single pharmaceutically acceptable formulation or in the form of separate, individual components, for example in the form of a kit comprising an immunogen component comprising the nucleotide sequence encoding an immunogen, and an adjuvant component comprising the 1H-imidazo[4,5-c]quinolin-4-amine, wherein the two components are for separate, sequential or simultaneous administration. Preferably, the administration of the two components is substantially simultaneous.

The IH-imidazo[4,5-c]quinolin-4-amine derivative as referred to throughout the specification and the claims is preferably a compound defined by one of Formulas 1-VI below:

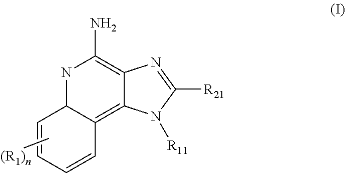

wherein

R₁₁ is selected from the group consisting of straight or branched chain alkyl, hydroxyalkyl, acyloxyalkyl, benzyl, (phenyl)ethyl and phenyl, said benzyl, (phenyl)ethyl or phenyl substituent being optionally substituted on the benzene ring by one or two moieties independently selected from the group consisting of alkyl of one to about four carbon atoms, alkoxy of one to about four carbon atoms and halogen, with the proviso that if said benzene ring is substituted by two of said moieties, then said moieties together contain no more than 6 carbon atoms; R₂₁ is selected from the group consisting of hydrogen, alkyl of one to about eight carbon atoms, benzyl, (phenyl)ethyl and phenyl, the benzyl, (phenyl)ethyl or phenyl substituent being optionally substituted on the benzene ring by one or two moieties independently selected from the group consisting of alkyl of one to about four carbon atoms, alkoxy of one to about four carbon atoms and halogen, with the proviso that when the benzene ring is substituted by two of said moieties, then the moieties together contain no more than 6 carbon atoms; and each R₁ is independently selected from the group consisting of hydrogen, alkoxy of one to about four carbon atoms, halogen and alkyl of one to about four carbon atoms, and n is an integer from 0 to 2, with the proviso that if n is 2, then said R₁₁ groups together contain no more than 6 carbon atoms;

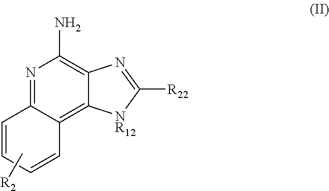

wherein

R₁₂ is selected from the group consisting of straight chain or branched chain alkenyl containing 2 to about 10 carbon atoms and substituted straight chain or branched chain alkenyl containing 2 to about 10 carbon atoms, wherein the substituent is selected from the group consisting of straight chain or branched chain alkyl containing 1 to about 4 carbon atoms and cycloalkyl containing 3 to about 6 carbon atoms; and cycloalkyl containing 3 to about 6 carbon atoms substituted by straight chain or branched chain alkyl containing 1 to about 4 carbon atoms; and R₂₂ is selected from the group consisting of hydrogen, straight chain or branched chain alkyl containing one to about eight carbon atoms, benzyl, (phenyl)ethyl and phenyl, the benzyl, (phenyl)ethyl or phenyl substituent being optionally substituted on the benzene ring by one or two moieties independently selected from the group consisting of straight chain or branched chain alkyl containing one to about four carbon atoms, straight chain or branched chain alkoxy containing one to about four carbon atoms, and halogen, with the proviso that when the benzene ring is substituted by two such moieties, then the moieties together contain no more than 6 carbon atoms; and each R₂ is independently selected from the group consisting of straight chain or branched chain alkoxy containing one to about four carbon atoms, halogen, and straight chain or branched chain alkyl containing one to about four carbon atoms, and n is an integer from zero to 2, with the proviso that if n is 2, then said R₂ groups together contain no more than 6 carbon atoms;

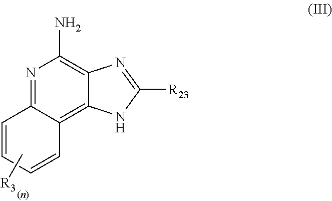

wherein

R₂₃ is selected from the group consisting of hydrogen, straight chain or branched chain alkyl of one to about eight carbon atoms, benzyl, (phenyl)ethyl and phenyl, the benzyl, (phenyl)ethyl or phenyl substituent being optionally substituted on the benzene ring by one or two moieties independently selected from the group consisting of straight chain or branched chain alkyl of one to about four carbon atoms, straight chain or branched chain alkoxy of one to about four carbon atoms, and halogen, with the proviso that when the benzene ring is substituted by two such moieties, then the moieties together—contain no more than 6 carbon atoms; and each R₅ is independently selected from the group consisting of straight chain or branched chain alkoxy of one to about four-carbon atoms, halogen, and 30 straight chain or branched chain alkyl of one to about four carbon atoms, and n is an integer from zero to 2, with the proviso that if n is 2, then said R₃ groups together contain no more than 6 carbon atoms;

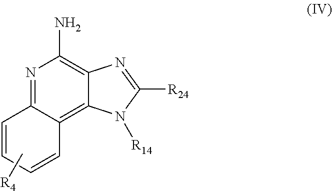

wherein

R₁₄ is —CHR_{A}R_{B} wherein R_{B} is hydrogen or a carbon-carbon bond, with the proviso that when R_{B} is hydrogen R_{A} is alkoxy of one to about four carbon atoms, hydroxyalkoxy of one to about four carbon atoms, 1-alkynyl of two to about ten carbon atoms, tetrahydropyranyl, alkoxyalkyl wherein the alkoxy moiety contains one to about four carbon atoms and the alkyl moiety contains one to about four carbon atoms, 2-, 3-, or 4-pyridyl, and with the further proviso that when R_{B} is a carbon-carbon bond R_{B} and R_{A} together form a tetrahydrofuranyl group optionally substituted with one or more substituents independently selected from the group consisting of hydroxy and hydroxyalkyl of one to about four carbon atoms; R₂₄ is selected from the group consisting of hydrogen, alkyl of one to about four carbon atoms, phenyl, and substituted phenyl wherein the substituent is selected from the group consisting of alkyl of one to about four carbon atoms, alkoxy of one to about four carbon atoms, and halogen; and R₄ is selected from the group consisting of hydrogen, straight chain or branched chain alkoxy containing one to about four carbon atoms, halogen, and straight chain or branched chain alkyl containing one to about four carbon atoms;

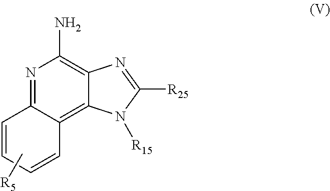

wherein

R₁₅ is selected from the group consisting of: hydrogen; straight chain or branched chain alkyl containing one to about ten carbon atoms and substituted straight chain or branched chain alkyl containing one to about ten carbon atoms, wherein the substituent is selected from the group consisting of cycloalkyl containing three to about six carbon atoms and cycloalkyl containing three to about six carbon atoms substituted by straight chain or branched chain alkyl containing one to about four carbon atoms; straight chain or branched chain alkenyl containing two to about ten carbon atoms and substituted straight chain or branched chain alkenyl containing two to about ten carbon atoms, wherein the substituent is selected from the group consisting of cycloalkyl containing three to about six carbon atoms and cycloalkyl containing three to about six carbon atoms substituted by straight chain or branched chain alkyl containing one to about four carbon atoms; hydroxyalkyl of one to about six carbon atoms; alkoxyalkyl wherein the alkoxy moiety contains one to about four carbon atoms and the alkyl moiety contains one to about six carbon atoms; acyloxyalkyl wherein the acyloxy moiety is alkanoyloxy of two to about four carbon atoms or benzoyloxy, and the alkyl moiety contains one to about six carbon atoms; benzyl; (phenyl)ethyl; and phenyl; said benzyl, (phenyl)ethyl or phenyl substituent being optionally substituted on the benzene ring by one or two moieties independently selected from the group consisting of alkyl of one to about four carbon atoms, alkoxy of one to about four carbon atoms, and halogen, with the proviso that when said benzene ring is substituted by two of said moieties, then the moieties together contain no more than six carbon atoms;

R₂₅ is

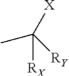

wherein

Rₓ and R_{y} are independently selected from the group consisting of hydrogen, alkyl of one to about four carbon atoms, phenyl, and substituted phenyl wherein the substituent is elected from the group consisting of alkyl of one to about four carbon atoms, alkoxy of one to about four carbon atoms, and halogen; X is selected from the group consisting of alkoxy containing one to about four carbon atoms, alkoxyalkyl wherein the alkoxy moiety contains one to about four carbon atoms and the alkyl moiety contains one to about four carbon atoms, haloalkyl of one to about four carbon atoms, alkylamido wherein the alkyl group contains one to about four carbon atoms, amino, substituted amino wherein the substituent is alkyl or hydroxyalkyl of one to about four carbon atoms, azido, alkylthio of one to about four carbon atoms; and R₅ is selected from the group consisting of hydrogen, straight chain or branched chain alkoxy containing one to about four carbon atoms, halogen, and straight chain or branched chain alkyl containing one to about four carbon atoms; or a pharmaceutically acceptable salt of any of the foregoing.

Preferred alkyl groups are C₁-C₄ alkyl, for example methyl, ethyl, propyl, 2-methylpropyl and butyl. Most preferred alkyl groups are methyl, ethyl and 2methyl-propyl. Preferred alkoxy groups are methoxy, ethoxy and ethoxymethyl.

The compounds recited above and methods for their preparation are disclosed in PCT patent application publication number WO 94/17043.

In instances where n can be zero, one, or two, n is preferably zero or one.

The substituents R₁-R₅ above are generally designated “benzo substituents” herein. The preferred benzo substituent is hydrogen.

The substituents R₁₁-R₁₅ above are generally designated “1-substituents” herein. The preferred 1-substituent is 2-methylpropyl or 2-hydroxy-2-methylpropyl.

The substituents R₂₁,-R₂₅ above are generally designated “2-substituents”, herein. The preferred 2-substituents are hydrogen, alkyl of one to about six carbon atoms, alkoxyalkyl wherein the alkoxy moiety contains one to about four carbon atoms and the alkyl moiety contains one to about four carbon atoms. Most preferably the 2-substituent is hydrogen, methyl, or ethoxymethyl.

Particularly preferred is when the 1H-imidazo[4,5-c]quinolin-4-amine is a compound defined by formula VI below:

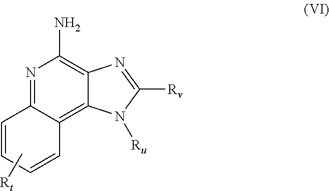

### Wherein

Rₜ is selected from the group consisting of hydrogen, straight chain or branched chain alkoxy containing one to about four carbon atoms, halogen, and straight chain or branched chain alkyl containing one to about four carbon atoms;

Rᵤ is 2-methylpropyl or 2-hydroxy-2-methylpropyl; and

Rᵥ is hydrogen, alkyl of one to about six carbon atoms, or alkoxyalkyl wherein the alkoxy moiety contains one to about four carbon atoms and the alkyl moiety contains one to about four carbon atoms; or physiologically acceptable salts of any of the foregoing, where appropriate.

In formula VI, Rₜ is preferably hydrogen, Rᵤ is preferably 2-methylpropyl or 2-hydroxy-2-methylpropyl, and Rv is preferably hydrogen, methyl or ethoxymethyl.

Preferred 1H-imidazo[4,5-c]quinolin-4-amines include the following:

1-(2-methylpropyl)-1H-imidazo[4,5-c]quinolin-4-amine (a compound of formula VI wherein Rₜ is hydrogen, Rᵤ is 2-methylpropyl and Rᵥ is hydrogen);

1-(2-hydroxy-2-methylpropyl)-2-methyl-1H-imidazo[4,5-c]quinolin-4-amine (a compound of formula VI wherein Rₜ is hydrogen, Rᵤ is 2-hydroxy-2-methylpropyl, and Rᵥ is methyl;

1-(2-hydroxy-2-methylpropyl)-1H-imidazo[4,5-c]quinolin-4-amine (a compound of formula VI wherein Rₜ is hydrogen, Rᵤ is 2-hydroxy-2-methylpropyl, and Rᵥ is hydrogen)

1-(2-hydroxy-2-methylpropyl)-2-ethoxymethyl-1-H-imidazo[4,5-c]quinolin-4-amine (a compound of formula VI wherein Rₜ is hydrogen, Rᵤ is 2-hydroxy-2-methylpropyl and Rᵥ is ethoxymethyl);

or physiologically acceptable salts thereof.

It is possible for the vaccination methods and compositions according to the present application to be adapted for protection or treatment of mammals against a variety of disease states such as, for example, viral, bacterial or parasitic infections, cancer, allergies and autoimmune disorders. Some specific examples of disorders or disease states which can be protected against or treated by using the methods or compositions according to the present invention, are as follows:

### Viral Infections

Hepatitis viruses A, B, C, D & E, HIV, herpes viruses 1,2, 6 & 7,-cytomegalovirus, varicella zoster, papilloma virus, Epstein Barr virus, influenza viruses, para-influenza viruses, adenoviruses, coxsakie viruses, picorna viruses, rotaviruses, respiratory syncytial viruses, pox viruses, rhinoviruses, rubella virus, papovirus, mumps virus, measles virus.

### Bacterial Infections

Mycobacteria causing TB and leprosy, pneumocci, aerobic gram negative bacilli, mycoplasma, staphyloccocal infections, streptococcal infections, salmonellae, chlamydiae.

### Parasitic

Malaria, leishmaniasis, trypanosomiasis, toxoplasmosis, schistosomiasis, filariasis,

### Cancer

Breast cancer, colon cancer, rectal cancer, cancer of the head and neck, renal cancer, malignant melanoma, laryngeal cancer, ovarian cancer, cervical cancer, prostate cancer.

### Allergies

Rhinitis due to house dust mite, pollen and other environmental allergens

### Autoimmune Disease

### Systemic Lupus Erythematosis

Preferably, the methods or compositions of the present invention are used to protect against or treat the viral disorders Hepatitis B, Hepatitis C, Human papilloma virus, Human immunodeficiency virus, or Herpes simplex virus; the bacterial disease TB; cancers of the breast, colon, ovary, cervix, and prostate; or the autoimmune diseases of asthma, rheumatoid arthritis and Alzheimer's

It is to be recognised that these specific disease states have been referred to by way of example only, and are not intended to be limiting upon the scope of the present invention.

The nucleotide sequences referred to in this application, which are to be expressed within a mammalian system, in order to induce an antigenic response, may encode for an entire protein, or merely a shorter peptide sequence which is capable of initiating an antigenic response. Throughout this specification and the appended claims, the phrase “antigenic peptide” or “immunogen” is intended to encompass all peptide or protein sequences which are capable of inducing an immune response within the animal concerned. Most preferably, however, the nucleotide sequence will encode for a full protein which is associated with the disease state, as the expression of full proteins within the animal system are more likely to mimic natural antigen presentation, and thereby evoke a full immune response. Some non-limiting examples of known antigenic peptides in relation to specific disease states include the following: Antigens which are capable of eliciting an immune response against a human pathogen, which antigen or antigenic composition is derived from HIV-1, (such as tat, nef, gp120 or gp160, gp40, p24, gag, env, vif, vpr, vpu, rev), human herpes viruses, such as gH, gL gM gB gC gK gE or gD or derivatives thereof or Immediate Early protein such as ICP27 , ICP 47, IC P 4, ICP36 from HSV1 or HSV2, cytomegalovirus, especially Human, (such as gB or derivatives thereof),

Epstein Barr virus (such as gp350 or derivatives thereof), Varicella Zoster Virus (such as gpI, II, III and IE63), or from a hepatitis virus such as hepatitis B virus (for example Hepatitis B Surface antigen or Hepatitis core antigen or pol), hepatitis C virus antigen and hepatitis E virus antigen, or from other viral pathogens, such as paramyxoviruses: Respiratory Syncytial virus (such as F and

G proteins or derivatives thereof), or antigens from parainfluenza virus, measles virus, mumps virus, human papilloma viruses (for example HPV6, 11, 16, 18, eg L1, L2, E1, E2, E3, E4, E5, E6, E7), flaviviruses (e.g. Yellow Fever Virus, Dengue Virus, Tick-borne encephalitis virus, Japanese Encephalitis Virus) or Influenza virus cells, such as HA, NP, NA, or M proteins, or combinations thereof), or antigens derived from bacterial pathogens such as Neisseria spp, including N. gonorrhea and N. meningitidis, eg, transferrin-binding proteins, lactoferrin binding proteins, PilC, adhesins); S. pyogenes (for example M proteins or fragments thereof, C5A protease, S. agalactiae, S. mutans; H. ducreyi; Moraxella spp, including M catarrhalis, also known as Branhamella catarrhalis (for example high and low molecular weight adhesins and invasins); Bordetella spp, including B. pertussis (for example pertactin, pertussis toxin or derivatives thereof, filamenteous hemagglutinin, adenylate cyclase, fimbriae), B. parapertussis and B. bronchiseptica; Mycobacterium spp., including M. tuberculosis (for example ESAT6, Antigen 85A, -B or -C, MPT 44, MPT59, MPT45, HSP10,HSP65, HSP70, HSP 75, HSP90, PPD 19 kDa [Rv3763], PPD 38 kDa [Rv0934]), M. bovis, M. leprae, M. avium, M. paratuberculosis, M. smegmatis; Legionella spp, including L. pneumophila; Escherichia spp, including enterotoxic E. coli (for example colonization factors, heat-labile toxin or derivatives thereof, heat-stable toxin or derivatives thereof), enterohemorragic E. coli, enteropathogenic E. coli (for example shiga toxin-like toxin or derivatives thereof); Vibrio spp, including V. cholera (for example cholera toxin or derivatives thereof); Shigella spp, including S. sonnei, S. dysenteriae, S. flexnerii; Yersinia spp, including Y. enterocolitica (for example a Yop protein), Y. pestis, Y. pseudotuberculosis; Campylobacter spp, including C. jejuni (for example toxins, adhesins and invasins) and C. coli; Salmonella spp, including S. typhi, S. paratyphi, S. choleraesuis, S. enteritidis; Listeria spp., including L. monocytogenes; Helicobacter spp, including H. pylori (for example urease, catalase, vacuolating toxin); Pseudomonas spp, including P. aeruginosa; Staphylococcus spp., including S. aureus, S. epidermidis; Enterococcus spp., including E. faecalis, E. faecium; Clostridium spp., including C. tetani (for example tetanus toxin and derivative thereof), C. botulinum (for example botulinum toxin and derivative thereof), C. difficile (for example clostridium toxins A or B and derivatives thereof); Bacillus spp., including B. anthracis (for example botulinum toxin and derivatives thereof); Corynebacterium spp., including C. diphtheriae (for example diphtheria toxin and derivatives thereof); Borrelia spp., including B. burgdorferi (for example OspA, OspC, DbpA, DbpB), B. garinii (for example OspA, OspC, DbpA, DbpB), B. afzelii (for example OspA, OspC, DbpA, DbpB), B. andersonii (for example OspA, OspC, DbpA, DbpB), B. hermsii; Ehrlichia spp., including E. equi and the agent of the Human Granulocytic Ehrlichiosis; Rickettsia spp, including R. rickettsii; Chlamydia spp., including C. trachomatis (for example MOMP, heparin-binding proteins), C. pneumoniae (for example MOMP, heparin-binding proteins), C. psittaci; Leptospira spp., including L. interrogans; Treponema spp., including T. pallidum (for example the rare outer membrane proteins), T. denticola, T. hyodysenteriae; or derived from parasites such as Plasmodium spp., including P. falciparum; Toxoplasma spp., including T. gondii (for example SAG2, SAG3, Tg34); Entamoeba spp., including E. histolytica; Babesia spp., including B. microti; Trypanosoma spp., including T. cruzi; Giardia spp., including G. lamblia; Leshmania spp., including L. major; Pneumocystis spp., including P. carinii; Trichomonas spp., including T. vaginalis; Schisostoma spp., including S. mansoni, or derived from yeast such as Candida spp., including C. albicans; Cryptococcus spp., including C. neoformans.

Other preferred specific antigens for M. tuberculosis are for example Rv2557, Rv2558, RPFs: Rv0837c, Rv1884c, Rv2389c, Rv2450, Rv1009, aceA (Rv0467), PstS1, (Rv0932), SodA (Rv3846), Rv2031c 16 kDal., Tb Ra12, Tb H9, Tb Ra35, Tb38-1, Erd 14, DPV, MTI, MSL, mTTC2 and hTCC1 (WO 99/51748). Proteins for M. tuberculosis also include fusion proteins and variants thereof where at least two, preferably three polypeptides of M. tuberculosis are fused into a larger protein. Preferred fusions include Ra12-TbH9-Ra35, Erd14-DPV-MTI, DPV-MTI-MSL, Erd14-DPV-MTI-MSL-mTCC2, Erd14-DPV-MTI-MSL, DPV-MTI-MSL-mTCC2, TbH9-DPV-MTI (WO 99/51748).

Most preferred antigens for Chlamydia include for example the High Molecular Weight Protein (HWMP) (WO 99/17741), ORF3 (EP 366 412), and putative membrane proteins (Pmps). Other Chlamydia antigens of the vaccine formulation can be selected from the group described in WO 99/28475.

Preferred bacterial vaccines comprise antigens derived from Streptococcus spp, including S. pneumoniae (PsaA, PspA, streptolysin, choline-binding proteins) and the protein antigen Pneumolysin (Biochem Biophys Acta, 1989, 67, 1007;

Rubins et al., Microbial Pathogenesis, 25, 337-342), and mutant detoxified derivatives thereof (WO 90/06951; WO 99/03884). Other preferred bacterial vaccines comprise antigens derived from Haemophilus spp., including H. influenzae type B (for example PRP and conjugates thereof), non typeable H. influenzae, for example OMP26, high molecular weight adhesins, P5, P6, protein D and lipoprotein D, and fimbrin and fimbrin derived peptides (U.S. Pat. No. 5,843,464) or multiple copy variants or fusion proteins thereof.

The antigens that may be used in the present invention may further comprise antigens derived from parasites that cause Malaria. For example, preferred antigens from Plasmodia falciparum include RTS,S and TRAP. RTS is a hybrid protein comprising substantially all the C-terminal portion of the circumsporozoite (CS) protein of P.falciparum linked via four amino acids of the preS2 portion of

Hepatitis B surface antigen to the surface (S) antigen of hepatitis B virus. Its full structure is disclosed in the International Patent Application No.

PCT/EP92/02591, published under Number WO 93/10152 claiming priority from UK patent application No. 9124390.7. When expressed in yeast RTS is produced as a lipoprotein particle, and when it is co-expressed with the S antigen from HBV it produces a mixed particle known as RTS,S. TRAP antigens are described in the International Patent Application No. PCT/GB89/00895, published under WO 90/01496. A preferred embodiment of the present invention is a Malaria vaccine wherein the antigenic preparation comprises a combination of the RTS, S and TRAP antigens. Other plasmodia antigens that are likely candidates to be components of a multistage Malaria vaccine are P. faciparum MSP1, AMA1, MSP3, EBA, GLURP, RAP1, RAP2, Sequestrin, PfEMP1, Pf332, LSA1, LSA3, STARP, SALSA, PfEXP1, Pfs25, Pfs28, PFS27/25, Pfs16, Pfs48/45, Pfs230 and their analogues in Plasmodium spp.

The invention contemplates the use of an anti-tumour antigen and be useful for the immunotherapeutic treatment of cancers. For example, tumour rejection antigens such as those for prostrate, breast, colorectal, lung, pancreatic, renal or melanoma cancers. Exemplary antigens include MAGE 1, 3 and MAGE 4 or other MAGE antigens such as disclosed in WO99/40188, PRAME, BAGE, Lage (also known as NY Eos 1) SAGE and HAGE (WO 99/53061) or GAGE (Robbins and Kawakami, 1996, Current Opinions in Immunology 8, pps 628-636; Van den Eynde et al., International Journal of Clinical & Laboratory Research (submitted 1997); Correale et al. (1997), Journal of the National Cancer Institute 89, p 293. Indeed these antigens are expressed in a wide range of tumour types such as melanoma, lung carcinoma, sarcoma and bladder carcinoma.

MAGE antigens for use in the present invention may be expressed as a fusion protein with an expression enhancer or an Immunological fusion partner. In particular, the Mage protein may be fused to Protein D from Heamophilus influenzae B. In particular, the fusion partner may comprise the first ⅓ of Protein D. Such constructs are disclosed in Wo99/40188. Other examples of fusion proteins that may contain cancer specific epitopes include bcr/abl fusion proteins.

In a preferred embodiment prostate antigens are utilised, such as Prostate specific antigen (PSA), PAP, PSCA (PNAS 95(4) 1735-1740 1998), PSMA or antigen known as Prostase.

Prostase is a prostate-specific serine protease (trypsin-like), 254 amino acid-long, with a conserved serine protease catalytic triad H-D-S and a amino-terminal pre-propeptide sequence, indicating a potential secretory function (P. Nelson, Lu Gan, C. Ferguson, P. Moss, R. Gelinas, L. Hood & K. Wand, “Molecular cloning and characterisation of prostase, an androgen-regulated serine protease with prostate restricted expression, In Proc. Natl. Acad. Sci. USA (1999) 96, 3114-3119). A putative glycosylation site has been described. The predicted structure is very similar to other known serine proteases, showing that the mature polypeptide folds into a single domain. The mature protein is 224 amino acids-long, with one A2 epitope shown to be naturally processed.

Prostase nucleotide sequence and deduced polypeptide sequence and homologs are disclosed in Ferguson, et al. (Proc. Natl. Acad. Sci. USA 1999, 96, 3114-3119) and in International Patent Applications No. WO 98/12302 (and also the corresponding granted patent U.S. Pat. No. 5,955,306), WO 98/20117 (and also the corresponding granted patents U.S. Pat. No. 5,840,871 and U.S. Pat. No. 5,786,148) (prostate-specific kallikrein) and WO 00/04149 (P703P).

The present invention provides antigens comprising prostase protein fusions based on prostase protein and fragments and homologues thereof (”derivatives“). Such derivatives are suitable for use in therapeutic vaccine formulations which are suitable for the treatment of a prostate tumours. Typically the fragment will contain at least 20, preferably 50, more preferably 100 contiguous amino acids as disclosed in the above referenced patent and patent applications.

A further preferred prostate antigen is known as P501S, sequence ID no 113 of WO98/37814. Immunogenic fragments and portions encoded by the gene thereof comprising at least 20, preferably 50, more preferably 100 contiguous amino acids as disclosed in the above referenced patent application, are contemplated. A particular fragment is PS108 (WO 98/50567).

Other prostate specific antigens are known from Wo98/37418, and WO/004149. Another is STEAP PNAS 96 14523 14528 7-12 1999.

Other tumour associated antigens useful in the context of the present invention include: Plu-1 J Biol. Chem 274 (22) 15633-15645, 1999, HASH-1, HasH-2, Cripto (Salomon et al Bioessays 199, 21 61-70,U.S. Pat. No. 5,654,140) Criptin U.S. Pat. No. 5,981,215. Additionally, antigens particularly relevant for vaccines in the therapy of cancer also comprise tyrosinase and survivin.

The present invention is also useful in combination with breast cancer antigens such as Muc-1, Muc-2, EpCAM, her 2/Neu, mammaglobin (U.S. Pat. No. 5,668,267) or those disclosed in WO/00 52165, WO99/33869, WO99/19479, WO 98/45328. Her 2 neu antigens are disclosed inter alia, in U.S. Pat. No. 5,801,005. Preferably the Her 2 neu comprises the entire extracellular domain (comprising approximately amino acid 1-645) or fragments thereof and at least an immunogenic portion of or the entire intracellular domain approximately the C terminal 580 amino acids . In particular, the intracellular portion should comprise the phosphorylation domain or fragments thereof. Such constructs are disclosed in WO00/44899. A particularly preferred construct is known as ECD PD a second is known as ECD PD. (See WO/00/44899.)

The her 2 neu as used herein can be derived from rat, mouse or human.

The vaccine may also contain antigens associated with tumour-support mechanisms (e.g. angiogenesis, tumour invasion) for example tie 2, VEGF.

Vaccines of the present invention may also be used for the prophylaxis or therapy of chronic disorders in addition to allergy, cancer or infectious diseases. Such chronic disorders are diseases such as asthma, atherosclerosis, and Alzheimers and other auto-immune disorders. Vaccines for use as a contraceptive may also be considered.

Antigens relevant for the prophylaxis and the therapy of patients susceptible to or suffering from Alzheimer neurodegenerative disease are, in particular, the N terminal 39-43 amino acid fragment (ABthe amyloid precursor protein and smaller fragments. This antigen is disclosed in the International Patent Application No. WO 99/27944—(Athena Neurosciences).

Potential self-antigens that could be included as vaccines for auto-immune disorders or as a contraceptive vaccine include: cytokines, hormones, growth factors or extracellular proteins, more preferably a 4-helical cytokine, most preferably IL13. Cytokines include, for example, IL1, IL2, IL3, IL4, IL5, IL6, IL7, IL8, IL9, IL10, IL11, IL12, IL13, IL14, IL15, IL16, IL17, IL18, IL20, IL21, TNF, TGF, GMCSF, MCSF and OSM. 4-helical cytokines include IL2, IL3, IL4, IL5, IL13, GMCSF and MCSF. Hormones include, for example, luteinising hormone (LH), follicle stimulating hormone (FSH), chorionic gonadotropin (CG), VGF, GHrelin, agouti, agouti related protein and neuropeptide Y. Growth factors include, for example, VEGF.

The vaccines of the present invention are particularly suited for the immunotherapeutic treatment of diseases, such as chronic conditions and cancers, but also for the therapy of persistent infections. Accordingly the vaccines of the present invention are particularly suitable for the immunotherapy of infectious diseases, such as Tuberculosis (TB), HIV infections such as AIDS and Hepatitis B (HepB) virus infections.

In a particularly preferred embodiment the nucleic acid encodes one or more of the following antigens:

HBV—PreS1 PreS2 and Surface env proteins, core and pol

HIV—gp120 gp40, gp160, p24, gag, pol, env, vif, vpr, vpu, tat, rev, nef

Papilloma—E1, E2, E3, E4, E5, E6, E7, E8, L1, L2

HSV—gL, gH, gM, gB, gC, gK, gE, gD, ICP47, ICP36, ICP4

Influenza—haemaggluttin, nucleoprotein

TB—Mycobacterial super oxide dismutase, 85A, 85B, MPT44, MPT59, MPT45, HSP10, HSP65, HSP70, HSP90, PPD 19 kDa Ag, PPD 38 kDa Ag.

The nucleotide sequence may be RNA or DNA including genomic DNA, synthetic DNA or cDNA. Preferably the nucleotide sequence is a DNA sequence and most preferably, a cDNA sequence. In order to obtain expression of the antigenic peptide within mammalian cells, it is necessary for the nucleotide sequence encoding the antigenic peptide to be presented in an appropriate vector system. By “appropriate vector” as used herein is meant any vector that will enable the antigenic peptide to be expressed within a mammal in sufficient quantities to evoke an immune response.

For example, the vector selected may comprise a plasmid, promoter and polyadenylation/ transcriptional termination sequence arranged in the correct order to obtain expression of the antigenic peptides. The construction of vectors which include these components and optionally other components such as enhancers, restriction enzyme sites and selection genes, such as antibiotic resistance genes, is well known to persons skilled in the art and is explained in detail in Maniatis et al “Molecular Cloning: A Laboratory Manual”, Cold Spring Harbour Laboratory, Cold Spring Harbour Press, Vols 1-3, 2^{nd} Edition, 1989.

As it is preferred to prevent the plasmids replicating within the mammalian host and integrating within the chromosomal DNA of the animal, the plasmid will preferably be produced without an origin of replication that is functional in eukaryotic cells.

The methods and compositions according to the present invention can be used in relation to prophylactic or treatment procedures of all mammals including, for example, domestic animals, laboratory animals, farm animals, captive wild animals and, most preferably, humans.

The present inventors have demonstrated that 1H-imidazo[4,5-c]quinolin-4-amine derivatives when used as adjuvants in DNA vaccination are capable of enhancing both Th1 and Th2 cytokine profiles. The term adjuvant or adjuvant component as used herein is intended to convey that the derivatives or component comprising the derivatives act to enhance and/or alter the body's response to an immunogen in a desired fashion. So, for example, an adjuvant may be used to shift an immune response to a predominately Th1 response, or to increase both types of responses.

A preferential inducer of a TH1 type of immune response enables a cell mediated response to be generated. High levels of Th1-type cytokines tend to favour the induction of cell mediated immune responses to the given antigen, whilst high levels of Th2-type cytokines tend to favour the induction of humoral immune responses to the antigen.

It is important to remember that the distinction of Th1 and Th2-type immune response is not absolute. In reality an individual will support an immune response which is described as being predominantly Th1 or predominantly Th2. However, it is often convenient to consider the families of cytokines in terms of that described in murine CD4 + ve T cell clones by Mosmann and Coffman (Mosmann, T. R. and Coffman, R. L. (1989) TH1 and TH2 cells: different patterns of lymphokine secretion lead to different functional properties. Annual Review of Immunology, 7, p 145-173). Traditionally, Th1-type responses are associated with the production of the INF-γ and IL-2 cytokines by T-lymphocytes. Other cytokines often directly associated with the induction of Th1-type immune responses are not produced by T-cells, such as IL-12. In contrast, Th2-type responses are associated with the secretion of IL-4, IL-5, IL-6, IL-10.

The immunogen component comprising a vector which comprises the nucleotide sequence encoding an antigenic peptide can be administered in a variety of manners. It is possible for the vector to be administered in a naked form (that is as naked nucleotide sequence not in association with liposomal formulations, with viral vectors or transfection facilitating proteins) suspended in an appropriate medium, for example a buffered saline solution such as PBS and then injected intramuscularly, subcutaneously, intraperitonally or intravenously, although some earlier data suggests that intramuscular or subcutaneous injection is preferable (Brohm et al Vaccine 16 No. 9/10 pp 949-954 (1998), the disclosure of which is included herein in its entirety by way of reference). It is additionally possible for the vectors to be encapsulated by, for example, liposomes or within polylactide co-glycolide (PLG) particles (25) for administration via the oral, nasal or pulmonary routes in addition to the routes detailed above.

It is also possible, according to a preferred embodiment of the invention, for intradermal administration of the immunogen component, preferably via use of gene-gun (particularly particle bombardment) administration techniques. Such techniques may involve coating of the immunogen component on to gold beads which are then administered under high pressure into the epidermis, such as, for example, as described in Haynes et al J. Biotechnology 44: 37-42 (1996).

The vectors which comprise the nucleotide sequences encoding antigenic peptides are administered in such amount as will be prophylactically or therapeutically effective. The quantity to be administered, is generally in the range of one picogram to 1 milligram, preferably 1 picogram to 10 micrograms for particle-mediated delivery, and 10 micrograms to 1 milligram for other routes of nucleotide per dose. The exact quantity may vary considerably depending on the species and weight of the mammal being immunised, the route of administration, the potency and dose of the 1H-imidazo-[4,5-c]quinolin derivative, the nature of the disease state being treated or protected against, the capacity of the subject's immune system to produce an immune response and the degree of protection or therapeutic efficacy desired. Based upon these variables, a medical or veterinary practitioner will readily be able to determine the appropriate dosage level.

It is possible for the immunogen component comprising the nucleotide sequence encoding the antigenic peptide, to be administered on a once off basis or to be administered repeatedly, for example, between 1 and 7 times, preferably between 1 and 4 times, at intervals between about 1 day and about 18 months. Once again, however, this treatment regime will be significantly varied depending upon the size and species of animal concerned, the disease which is being treated/protected against, the amount of nucleotide sequence administered, the route of administration, the potency and dose of 1H-imidazo[4,5-c]quinolin-4-amine derivatives selected and other factors which would be apparent to a skilled veterinary or medical practitioner.

The adjuvant component specified herein can similarly be administered via a variety of different administration routes, such as for example, via the oral, nasal, pulmonary, intramuscular, subcutaneous, intradermal or topical routes. Preferably, the component is administered via the intradermal or topical routes. This administration may take place between about 14 days prior to and about 14 days post administration of the nucleotide sequence, preferably between about 1 day prior to and about 3 days post administration of the nucleotide sequence. Most preferred is when the adjuvant component is administered substantially simultaneously with the administration of the nucleotide sequence. By “substantially simultaneous” what is meant is that administration of the adjuvant component is preferably at the same time as administration of the nucleotide sequence, or if not, at least within a few hours either side of nucleotide sequence administration. In the most preferred treatment protocol, the adjuvant component will be administered substantially simultaneously to administration of the nucleotide sequence. Obviously, this protocol can be varied as necessary, in accordance with the type of variables referred to above.

Once again, depending upon such variables, the dose of administration of the derivative will also vary, but may, for example, range between about 0.1 mg per kg to about 100 mg per kg, where “per kg” refers to the body weight of the mammal concerned. This administration of the 1H-imidazo[4,5-c]quinolin-4-amine derivative would preferably be repeated with each subsequent or booster administration of the nucloetide sequence. Most preferably, the administration dose will be between about 1 mg per kg to about 50 mg per kg.

While it is possible for the adjuvant component to comprise only 1 H-imidazo[4,5-c]quinolin-4-amine derivatives to be administered in the raw chemical state, it is preferable for administration to be in the form of a pharmaceutical formulation. That is, the adjuvant component will preferably comprise the 1H-imidazo[4,5-c]quinolin-4-amine combined with one or more pharmaceutically or veterinarily acceptable carriers, and optionally other therapeutic ingredients. The carrier(s) must be “acceptable” in the sense of being compatible with other ingredients within the formulation, and not deleterious to the recipient thereof. The nature of the formulations will naturally vary according to the intended administration route, and may be prepared by methods well known in the pharmaceutical art. All methods include the step of bringing into association a 1H-imidazo[4,5-c]quinolin-4-amine derivative with an appropriate carrier or carriers. In general, the formulations are prepared by uniformly and intimately bringing into association the derivative with liquid carriers or finely divided solid carriers, or both, and then, if necessary, shaping the product into the desired formulation. Formulations of the present invention suitable for oral administration may be presented as discrete units such as capsules, cachets or tablets each containing a pre-determined amount of the active ingredient; as a powder or granules; as a solution or a suspension in an aqueous liquid or a non-aqueous liquid; or as an oil-in-water liquid emulsion or a water-in-oil emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent.

The tablets may optionally be coated or scored and may be formulated so as to provide slow or controlled release of the active ingredient.

Formulations for injection via, for example, the intramuscular, intraperitoneal, or subcutaneous administration routes include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampoules and vials, and may be stored in a freeze-dried (lyophilised) condition requiring only the addition of the sterile liquid carrier, for example, water for injections, immediately prior to use. Extemporaneous injection solutions and suspensions may be prepared from sterile powders, granules and tablets of the kind previously described. Formulations suitable for pulmonary administration via the buccal or nasal cavity are presented such that particles containing the active ingredient, desirably having a diameter in the range of 0.5 to 7 microns, are delivered into the bronchial tree of the recipient. Possibilities for such formulations are that they are in the form of finely comminuted powders which may conveniently be presented either in a piercable capsule, suitably of, for example, gelatine, for use in an inhalation device, or alternatively, as a self-propelling formulation comprising active ingredient, a suitable liquid propellant and optionally, other ingredients such as surfactant and/or a solid diluent. Self-propelling formulations may also be employed wherein the active ingredient is dispensed in the form of droplets of a solution or suspension. Such self-propelling formulations are analogous to those known in the art and may be prepared by established procedures. They are suitably provided with either a manually-operable or automatically functioning valve having the desired spray characteristics; advantageously the valve is of a metered type delivering a fixed volume, for example, 50 to 100 μL, upon each operation thereof.

In a further possibility, the adjuvant component may be in the form of a solution for use in an atomiser or nebuliser whereby an accelerated airstream or ultrasonic agitation is employed to produce a find droplet mist for inhalation.

Formulations suitable for intranasal administration generally include presentations similar to those described above for pulmonary administration, although it is preferred for such formulations to have a particle diameter in the range of about 10 to about 200 microns, to enable retention within the nasal cavity. This may be achieved by, as appropriate, use of a powder of a suitable particle size, or choice of an appropriate valve. Other suitable formulations include coarse powders having a particle diameter in the range of about 20 to about 500 microns, for administration by rapid inhalation through the nasal passage from a container held close up to the nose, and nasal drops comprising about 0.2 to 5% w/w of the active ingredient in aqueous or oily solutions. In one embodiment of the invention, it is possible for the vector which comprises the nucleotide sequence encoding the antigenic peptide to be administered within the same formulation as the 1H-imidazo[4,5-c]quinolin-4-amine derivative. Hence in this embodiment, the immunogenic and the adjuvant component are found within the same formulation.

In a preferred embodiment the adjuvant component is prepared in a form suitable for gene-gun administration, and is administered via that route substantially simultaneous to administration of the nucleotide sequence. For preparation of formulations suitable for use in this manner, it may be necessary for the 1H-imidazo[4,5-c]quinolin-4-amine derivative to be lyophilised and adhered onto, for example, gold beads which are suited for gene-gun administration.

In an alternative embodiment, the adjuvant component may be administered as a dry powder, via high pressure gas propulsion. This will preferably be substantially simultaneous to administration of the nucleotide sequence.

Even if not formulated together, it may be appropriate for the adjuvant component to be administered at or about the same administration site as the nucleotide sequence.

Other details of pharmaceutical preparations can be found in Remington's Pharmaceutical Sciences, Mack Publishing Company, Easton, Pa. (1985), the disclosure of which is included herein in its entirety, by way of reference.

The present invention will now be described further, with reference to the following non-limiting examples:

### Examples

1. Imiquimod Increases the Magnitude of the Cytotoxic T Cell Response to a Nucleic Acid Vaccine.

### Construction of Plasmids and DNA Preparation

The plasmids used are based upon pVAC1, obtained from Michelle Young, GlaxoWellcome, UK, a modification of the mammalian expression vector, pCl, (Promega), where the multiple cloning site, from EcoRI to Bst ZI, has been replaced by the EMCV IRES sequence flanked 5′ by unique Nhe I, Rsr II and Xho I and 3′ by unique Pac I, Asc I and Not I restriction enzyme sites.

An influenza nucleoprotein expression plasmid, pVAC1.PR, was constructed by ligating PCR amplified cDNA encoding nucleoprotein of influenza A virus strain PR/8/34 from pAR501, (a gift from Dr. D. Kiossis, NIMR, London, UK), into the expression vector pVAC1.

Plasmid DNA was propagated in E. coli, and prepared using plasmid purification kits (QIAGEN Ltd, Crawley, UK), and stored at −20° C. at approximately 1 mg plasmid DNA/ml in 10 mM Tris/EDTA buffer.

### Preparations of Cartridges for DNA Immunisation

Preparation of cartridges for the Accell gene transfer device was as previously described (Eisenbraun et al DNA and Cell Biology, 1993 Vol 12 No 9 pp 791-797; Pertner et al). Briefly, plasmid DNA was coated onto 2 μm gold particles (DeGussa Corp., South Plainfield, N.J., USA) and loaded into Tefzel tubing, which was subsequently cut into 1.27 cm lengths to serve as cartridges and stored desiccated at 4° C. until use. In a typical vaccination, each cartridge contained 0.5 mg gold coated with ˜0.05 μg pVAC1.PR with empty vector (pVAC1) added to provide a total of 0.5 μg DNA/cartridge.

### Immunisations

To examine whether imiquimod could increase the cytotoxic T cell response generated by nucleic acid vaccination, pVAC1.PR was administered by particle mediated gene transfer (0.05 μg/cartridge) into the skin of mice. Plasmid was delivered to the shaved target site of abdominal skin of C57B1/6 mice (purchased from Charles River United Kingdom Ltd, Margate, UK) from two cartridges using the Accell gene transfer device at 500 lb/in2 (McCabe WO 95/19799).) Immediately following vaccination imiquimod (prepared as a suspension in vehicle which comprised 0.3%(w/v) methylcellulose and 0.1% (v/v) Tween in sterile water) was administered by a single subcutaneous injection (0.05 ml/10 g body weight formulated to provide a dose of 30 mg/kg)) at the immunisation site. Plasmid and imiquimod controls were empty vector (pVAC1) and vehicle, respectively.

### Cytotoxic T Cell Responses

The cytotoxic T cell response was assessed by CD8+ T cell-restricted IFN-γ ELISPOT assay of splenocytes collected one week later. Mice were killed by cervical dislocation and spleens were collected into ice-cold PBS. Splenocytes were teased out into phosphate buffered saline (PBS) followed by lysis of red blood cells (1 minute in buffer consisting of 155 mM NH₄Cl, 10 mM KHCO₃, 0.1 mM EDTA). After two washes in PBS to remove particulate matter the single cell suspension was aliquoted into ELISPOT plates previously coated with capture IFN-γ antibody and stimulated with CD8-restricted cognate peptide. After overnight culture, IFN-γ producing cells were visualised by application of anti-murine IFN-γ-biotin labelled antibody (Pharmingen) followed by streptavidin—conjugated alkaline phosphatase and quantitated using image analysis.

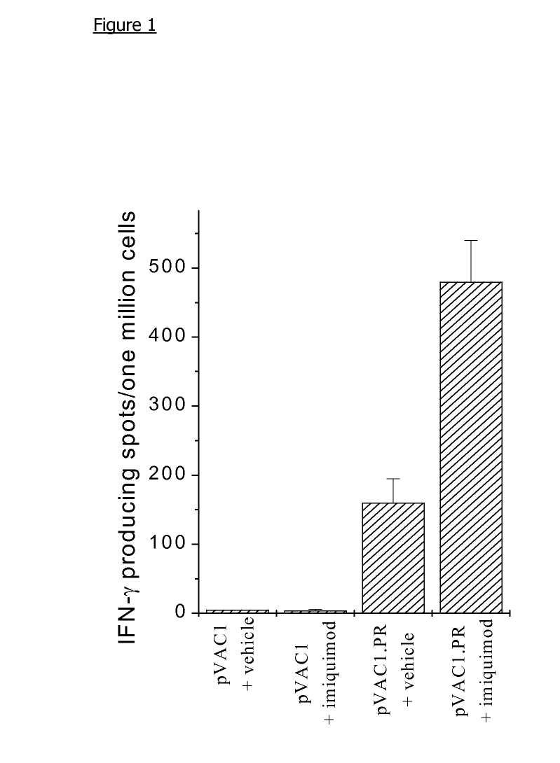

The results of this experiment (FIG. 1) show that the number of cytotoxic T cells in the spleens of mice treated with the combination of pVAC1.PR and imiquimod was 3 times greater than with pVAC1.PR+ vehicle alone. No difference was seen between the control plasmid (pVAC1)+imiquimod or vehicle groups indicating that the effect of imiquimod was antigen-restricted. These results clearly show that imiquimod is a potent adjuvant for nucleic acid vaccination.

2. Imiquimod Increases the Magnitude of the CD4+ T Cell Response to a Nucleic Acid Vaccine

### Plasmids, DNA and Cartridge Preparation

A chicken ovalbumin expression plasmid, pVAC1.OVA was constructed by ligating PCR amplified cDNA encoding chicken ovalbumin from pUGOVA (a gift from Dr. F. Carbone) into the expression vector pVAC1 and cartridges were prepared (as described in example 1 above).

### Mice and Immunisations

Male or female D0.11.10 transgenic mice (6-10 weeks old) were bred in our specific pathogen-free animal breeding facilities at Bury Green Farm. The transgene that these mice express is the T cell receptor (TCR) specific for a chicken ovalbumin peptide residue (residues 323-339; OVA peptide) bound to MHC-II molecule (I-A^{d}). The monoclonal antibody, KJ1-26 which specifically recognises this TCR is used for identification of TCR-transgenic T cells. Examination of a number of these mice shows that a large proportion (40-65%) of the CD4⁺ T cells are KJ1-26⁺, although a very small population of CD4⁻ CD8+ KJ1.26⁺ T cells are also present (Pape, et al Immunological Reviews (1997) 156: 67-78).

Balb/c mice were purchased from Charles River United Kingdom Ltd. (Margate, UK).

CD4+ T cell responses were examined using an adoptive transfer model which enhances the sensitivity of the immune parameters to be measured. Here, T cells which specifically recognise a peptide sequence from ovalbumin protein were adoptively transferred from transgenic into na-ve wild-type mice before immunisation. Briefly, 24 hours before immunisation, D0.11.10 splenocytes were adoptively transferred into Balb/c mice at 6-8 weeks of age. Splenocytes were prepared as in example 1 above. Cells were then adoptively transferred into the lateral tail vein by injection of 100 μl (i.e. 25×10⁶ splenocytes/mouse).

Mice were subsequently immunised by particle mediated gene transfer with a plasmid encoding ovalbumin (pVAC1.OVA; 0.5 μg/cartridge) and imiquimod (30 mg/kg), or with control plasmid or vehicle, as in example 1.

### CD4 T Cell Responses

Mice were killed by cervical dislocation and inguinal and periaortic lymph nodes were collected and prepared as for splenocytes (described in example 1), except that the red blood cell lysis step was omitted.

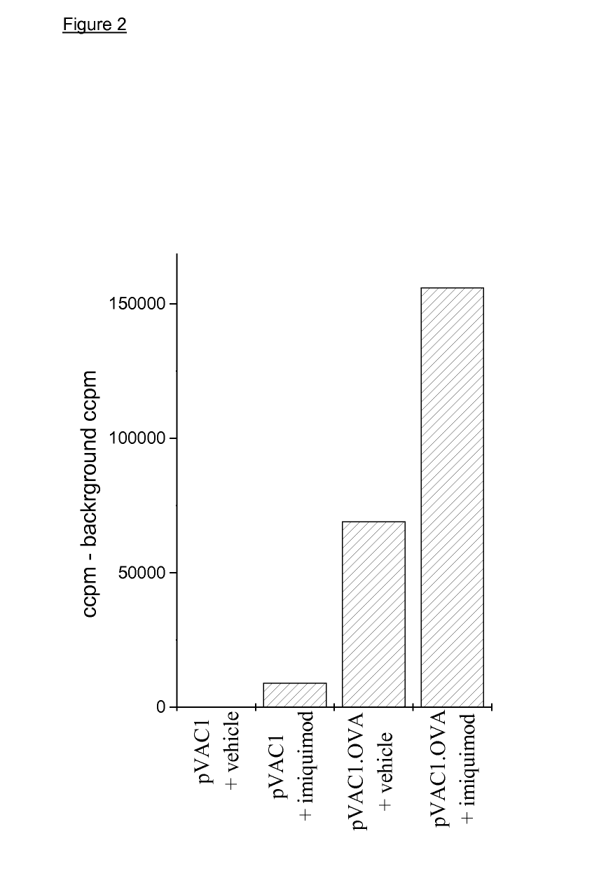

To measure the clonal expansion of CD4+ T cells following immunisation, proliferation of ovalbumin-specific T cells following re-challenge in vitro with cognate ovalbumin peptide, was assessed in lymph node preparations collected 3 days after immunisation. Proliferation, assessed by up take of tritiated thymidine by proliferating cells, was more than twice that with the combination of pVAC1.OVA+imiquimod compared with pVAC1.OVA+vehicle (FIG. 2). No difference was seen between the control plasmid (pVAC1)+imiquimod or vehicle groups indicating that the effect of imiquimod was antigen-restricted.

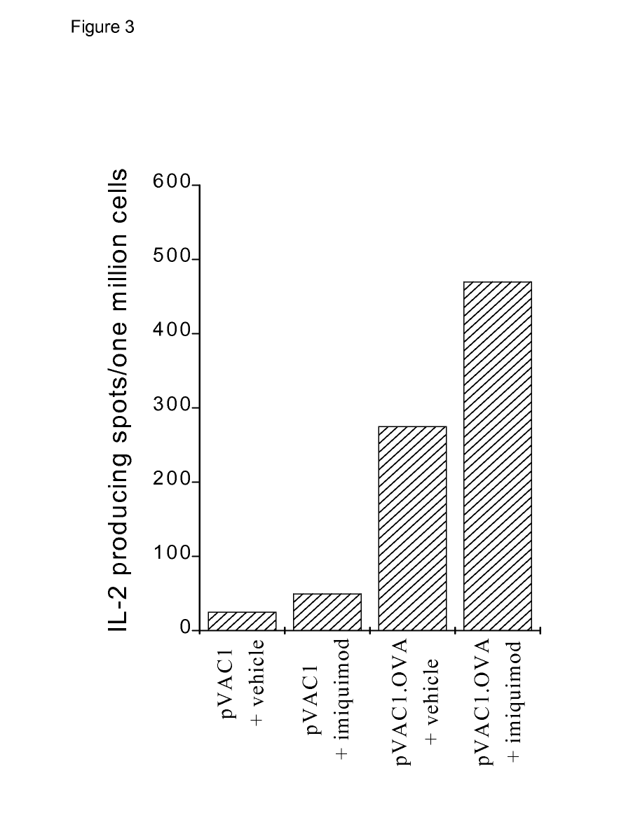

A substantial increase in activation of CD4 T cells, assessed by IL-2 ELISPOT (example 1, above) on lymph node cells collected 6 days after immunisation (FIG. 3), was found for the combination of pVAC1.OVA+imiquimod compared with pVAC1.OvA alone (70% increase).

These results show that the adjuvant effect of imiquimod in vivo extends to both (ie. CD8+ cytotoxic and CD4+ helper) T cell populations. They also confirm the potency of the adjuvant effect of imiquimod for nucleic acid vaccination.

3. Imiquimod Induces Both Th1 and Th2 Responses to a Nucleic Acid Vaccine.

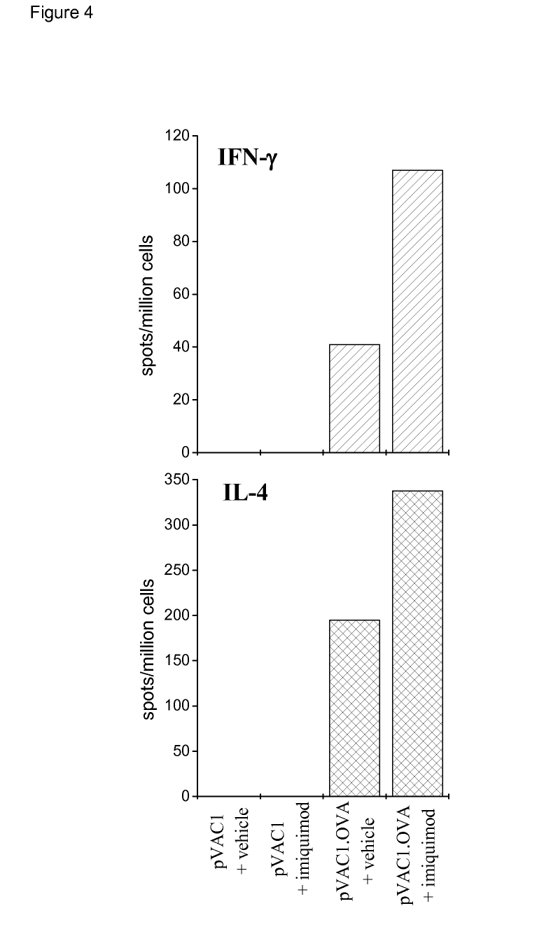

Th CD4+ subsets were assessed using the adoptive transfer model (example 2, above). IFN-γ-producing (Th1) and IL-4-producing (Th2) cells were assayed by ELISPOT (example 1, above). Imiquimod induced an increase in both cytokine-producing cells, although the increase was more substantial for IFN-γ compared with IL-4 (FIG. 4). No cytokine producing cells were detected in empty vector immunised controls. The bias towards IFN-γ-producing cells, indicates that imiquimod not only acts as an adjuvant for nucleic acid vaccination but that it preferentially induces a Th1 type of response, the latter being substantiated by example 1 above.

4. Effect of Imiquimod on Humoral Immune Responses

To examine whether imiquimod increases the humoral response generated by nucleic acid vaccination, a plasmid encoding an antigen (eg. nucleoprotein from influenza virus) is administered by PMGT into the skin of mice. A primary immunisation is followed by one or more boost immunisations, with at least 4 weeks between each immunisation. Before and/or immediately following each immunisation imiquimod is administered by a single subcutaneous injection at the immunisation site. Plasmid and imiquimod controls are empty vector (ie. without antigen) and vehicle, respectively. Blood samples are collected from the tail vein 1 to 3 days prior to, and at intervals after, immunisation. Serum is separated and stored at −20° C. for subsequent antibody analysis.

The humoral response is assessed by measuring antigen-specific whole IgG antibody levels (eg. nucleoprotein from influenza virus) in the serum samples collected after primary and boost immunisation. Microtitre plates (Nunc Immunoplate F96 maxisorp, Lifet Technologies) are coated with 10 μg/ml antigen by overnight incubation at 4° C. and washed 4 times with washing buffer (PBS containing 5% Tween 20 and 0.1% sodium azide). This is followed by a 1 hour incubation at 20° C. with serum samples serially diluted in blocking buffer. After 4 further washes (as above) to remove unbound antibody, plates are incubated for 1 hour with peroxidase conjugated anti-mouse IgG antibody (Southern Biotechnology) diluted in blocking buffer. The amount of bound antibody is determined after 4 further washes (as above) followed by addition of TMB substrate solution (T-8540-Sigma). After 30 minutes at 20° C. protected from light, the reaction is stopped with 1M sulphuric acid and absorbance read at 450 nm. Titres are defined as the highest dilution to reach an OD of 0.2. An increase in antibody levels in immunised mice treated with imiqimod over those immunised without imiquimod indicates an enhancing effect of imiquimod on humoral responses.

5. Imiquimod Enhances Immune Response to HIV Antigens

### Plasmids, DNA and Cartridge Preparation

A plasmid expressing the Gag and Nef antigens (ie.WRG7077Gag/Nef) was constructed based on WRG7077. The original WRG7077 plasmid was constructed by replacing the beta-lactamase gene containing Eam11051-PstI fragment of pUC19 (available from Amersham Pharmacia Biotech UK Ltd., Amersham Place, Little Chalfont, Bucks, HP7 9NA) with an EcoRI fragment of pUC4K (Amersham-Pharmacia) containing the Kanamycin resistance gene, following blunt ending of both fragments using T4 DNA polymerase. The human Cytomegalovirus IE1 promoter/enhancer, intron A, was derived from plasmid JW4303 obtained from Dr Harriet Robinson, University of Massachussets, and inserted into the SalI site of pUC19 as a XhoI-Sal1 fragment, incorporating the bovine growth hormone polyadenylation signal. The Gag-Nef fusion was generated by PCR stitching of a truncated Nef with 195bp deleted from the 5′ end of the gene removing the first 65 amino acids, derived from HIV-1 strain 248A (Genbank Acc. No. L15518, a kink gift from G. Thompson), and p17p24 (Gag) from the plasmid pHXBAPr (Maschera et al., 1995) containing HIV-1 clade B strain HXB2 (Genbank Acc. No. K03455). The resulting Gag-Nef fusion was subsequently Ligated into WRG7077 as a NotI-BamHI fragment. Plasmid DNA and cartridge preparation was as described in example 1.

### Mice and Immunisations

Balb/c mice were immunised, and imiquimod prepared and administered, as described in example 1. Here mice received a primary immunisation followed by a boost immunisation 42 days later. Imiquimod (100 mg/kg) was administered at the boost only.

### Cytotoxic T Cell Responses

IFN-γ ELISPOT assays were performed as described (example 1), using CD8-restricted cognate peptides for the Gag and Nef antigens to stimulate splenocytes collected 5 days after the boost immunisation.

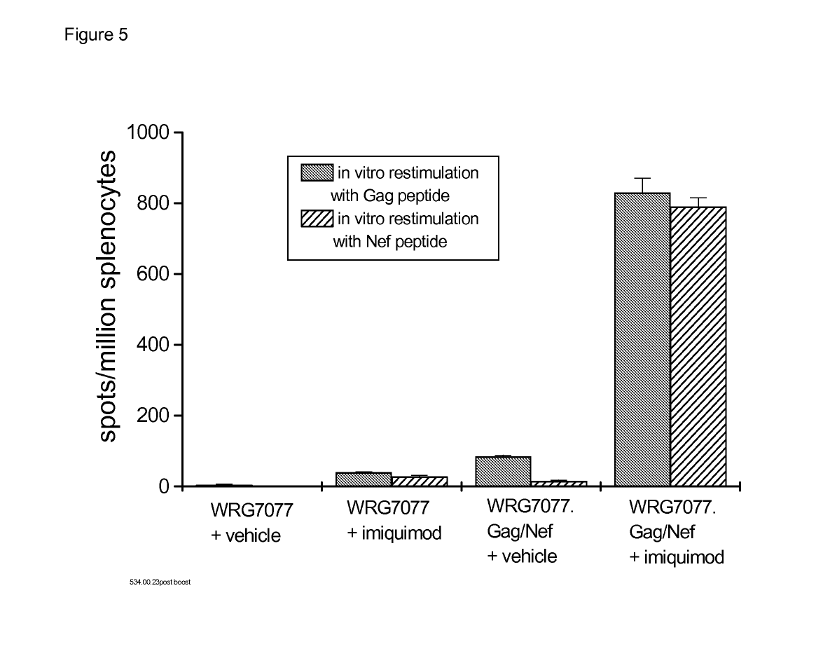

A substantial increase in cytotoxic T cells was observed when imiquimod was co-administered compared with the group that received plasmid with vehicle alone (10 fold and 100 fold for Gag and Nef, respectively) (FIG. 5). These findings suggest that the adjuvant effect of imiquimod is not antigen specific and exemplify efficacy with viral antigens, most specifically for HIV.

### 6. Analogues of Imiquimod are Effective Adjuvants for Nucleic Acid Vaccination.

Cartridges were prepared using the pVAC1.OVA plasmid, immunisations and T cell responses were as described in example 2. Analogues of imiquimod tested were 1-(2-hydroxy-2-methylpropyl)-2-ethoxymethyl-1-H-imidazo[4,5-c]quinolin-4-amine (ie. resiquimod), 1-(2-hydroxy-2-methylpropyl)-2-methyl-1H-imidazo[4,5-c]quinolin-4-amine and 1-(2,hydroxy-2-methylpropyl)-1H-imidazo[4,5-c]quinolin-4-amine.

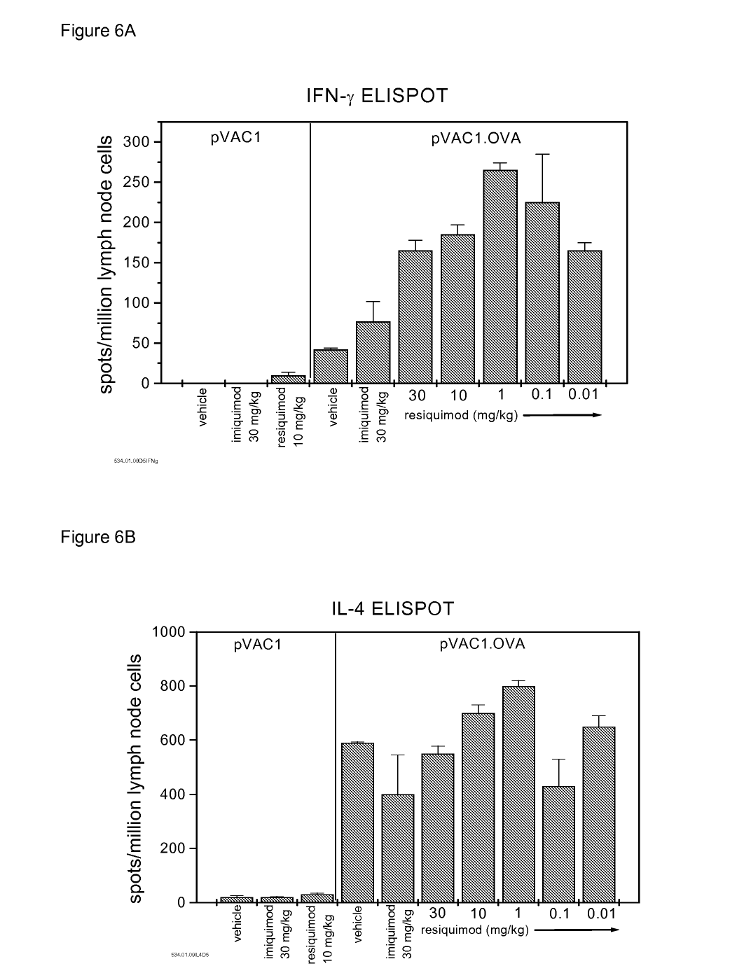

Resiquimod was tested over a broad range of doses, with 1 mg/kg apparently optimal. Here analysis of lymph node cells collected 5 days after immunisations showed a six fold increase of IFN-γ producing CD4 T cells, with only a modest effect on IL-4 producing CD4 T cells (FIG. 6), further supporting the Th1 biased effect observed in example 3.

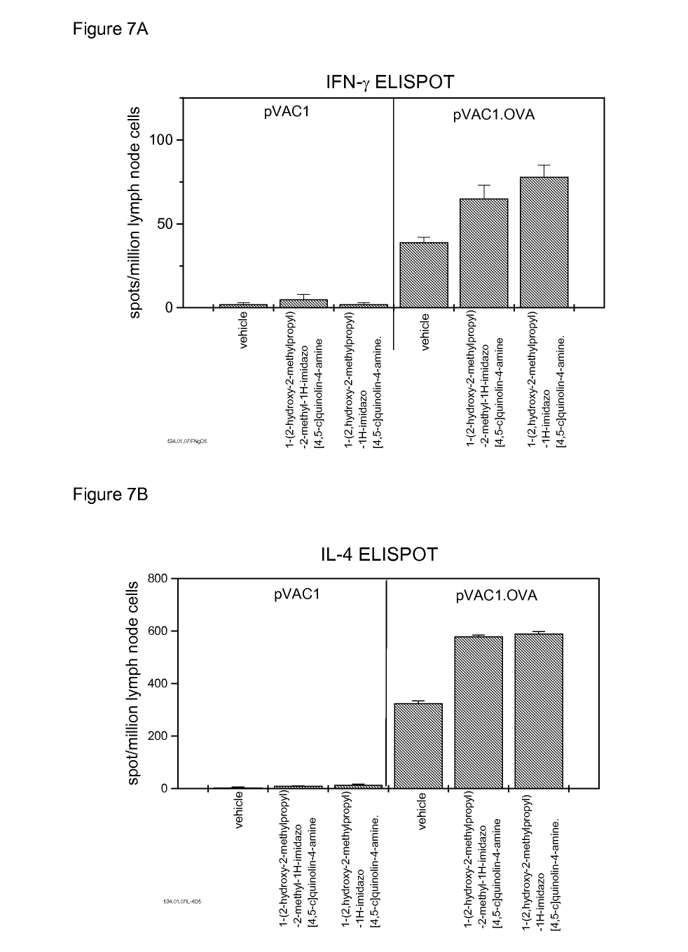

The effect on T cells 5 days after immunisation was investigated for the other two analogues given at the single dose of 30 mg/kg. Two fold increases in both IFN-γ and IL-4 producing CD4 T cells were induced by 1-(2-hydroxy-2-methylpropyl)-2-methyl-1H-imidazo[4,5-c]quinolin-4-amine. A similar effect was observed for 1-(2,hydroxy-2-methylpropyl)-1H-imidazo[4,5-c]quinolin-4-amine (FIG. 7).

Overall, these results in this example show that the adjuvant effects are not restricted to imiquimod alone, but extend to the broader range of compounds in this chemical class.

7. Topical Application is an Effective Route for Administration of Imiquimod with Nucleic Acid Vaccination.

Cartridges were prepared using the WRG7077Gag/Nef plasmid, PMID immunisations and T cell responses were as described in example 5. Here, imiquimod was administered subcutaneously at a dose of 30 mg/kg. An additional group was included where imiquimod was administered by application of 20 μl of a 5% (w/v)cream formulation (ie. Aldara) applied topically at the PMID site immediately after immunisation. Spleens were collected for analysis 6 and 11 days after the boost immunisation.

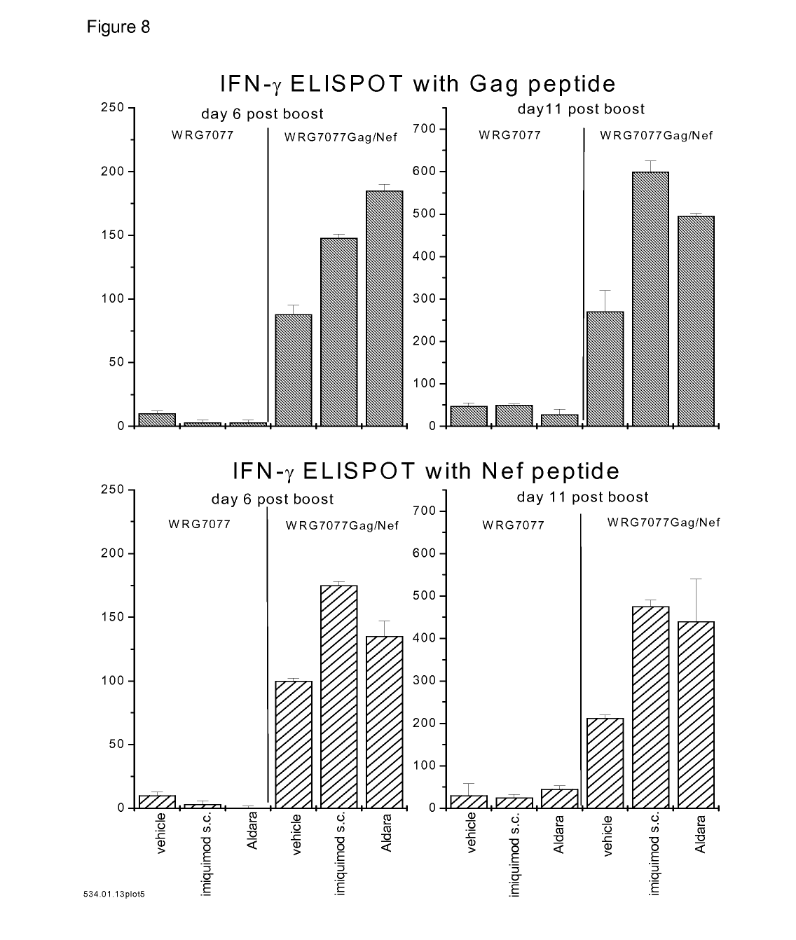

A substantial increase in cytotoxic T cells was observed when imiquimod was co-administered, either as s.c. injection or topically, compared with the group that received plasmid with s.c vehicle alone (ie. approximately 2 fold 11 days after boost) (FIG. 8). These findings show that topically applied imiquimod is an effective adjuvant for PMID and suggest that subcutaneous injection is a relevant surrogate route for topical application in investigations of adjuvants combined with PMID.

8. Imiquimod is an Effective Adjuvant for the Prevention of Tumour Growth.

Cartridges were prepared using the pVAC1.OVA plasmid (as described in example 2). Groups of 12 mice were immunised by PMID as described in example 5, except that cartridges contained 0.1 μg of DNA and imiquimod was administered subcutaneously at a dose of 30 mg/kg.

### Tumour Challenge

Two weeks after the boost, EG7-OVA cells were implanted by needle injection subcutaneously in the flank of each mouse (100,000 cells/mouse) and tumour growth monitored. The E.G7-OVA cells were originally generated by F. Carbone (Moore, M. W., Carbone, F. R. and Bevan, M. J., Cell, 54, 777-785, 1988) by transfecting the mouse ascites lymphoma lymphoblast cell line, EL4, to stably express chicken ovalbumin.

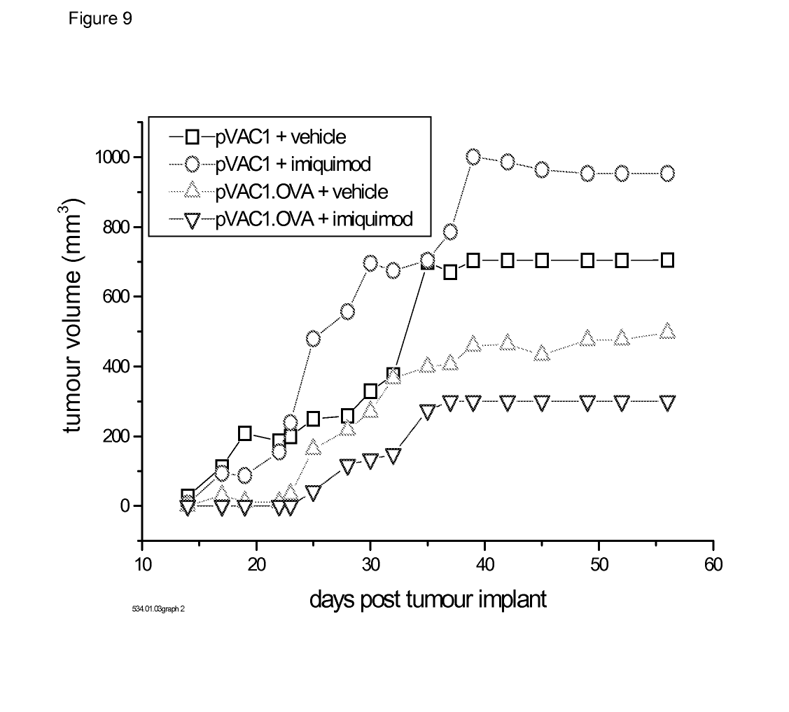

The appearance of palpable tumours was most delayed for mice immunised with pVAC1.OVA + imiquimod compared with pVAC1.OVA alone (FIG. 9). Palpable tumours appeared earlier and grew most rapidly for control groups of mice (ie.

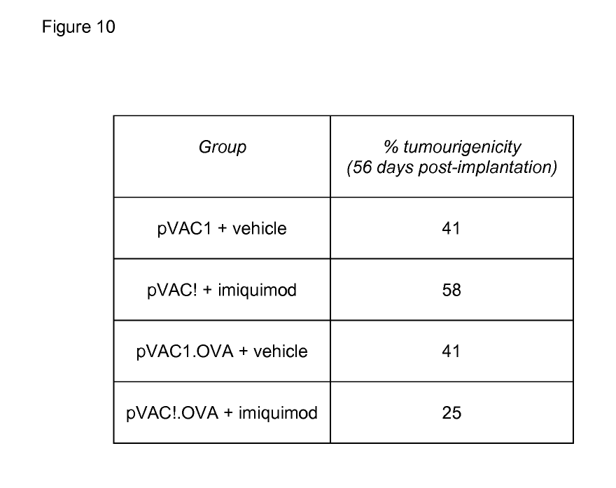

mice immunised with empty vector±imiquimod). Tumourigenicity was 25% for the mice immunised with pVAC1.OVA+imiquimod. In contrast, tumourigenicity was greater than 40% for all other groups (FIG. 10). These results show that imiquimod can improve the antitumour effect of PMID, and suggest that imiquimod will be an effective adjuvant with nucleic acid for treatment of disease.

1. A vaccine composition comprising (i) an adjuvant component comprising a 1H-imidazo[4,5-c]quinolin-4-amine derivative and (ii) an immunogen component comprising a nucleotide sequence encoding an antigenic peptide or protein associated with a disease state.

2. A kit comprising (1) an adjuvant component comprising a 1H-imidazo[4,5-c]quinolin-4-amine derivative and ii) an immunogen component comprising a nucleotide sequence encoding an antigenic peptide or protein associated with a disease state.

3. The kit according to claim 2 wherein the components are for substantially simultaneous administration.

4. The composition according to claim 1 wherein the components are within a single pharmaceutically acceptable formulation.

5. The composition according to claim 1 wherein the 1H-imidazo[4,5-c]quinolin-4-amine derivative is a compound defined by one of formulae l-VI: 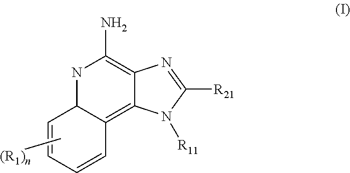
wherein
R₁₁ is selected from the group consisting of straight or branched chain alkyl, hydroxyalkyl, acyloxyalkyl, benzyl, (phenyl)ethyl and phenyl, said benzyl, (phenyl)ethyl or phenyl substituent being optionally substituted on the benzene ring by one or two moieties independently selected from the group consisting of alkyl of one to about four carbon atoms, alkoxy of one to about four carbon atoms and halogen, with the proviso that if said benzene ring is substituted by two of said moieties, then said moieties together contain no more than 6 carbon atoms; R₂₁ is selected from the group consisting of hydrogen, alkyl of one to about eight carbon atoms, benzyl, (phenyl)ethyl and phenyl, the benzyl, (phenyl)ethyl or phenyl substituent being optionally substituted on the benzene ring by one or two moieties independently selected from the group consisting of alkyl of one to about four carbon atoms, alkoxy of one to about four carbon atoms and halogen, with the proviso that when the benzene ring is substituted by two of said moieties, then the moieties together contain no more than 6 carbon atoms; and each R₁ is independently selected from the group consisting of hydrogen, alkoxy of one to about four carbon atoms, halogen and alkyl of one to about four carbon atoms, and n is an integer from 0 to 2, with the proviso that if n is 2, then said R₁₁ groups together contain no more than 6 carbon atoms; 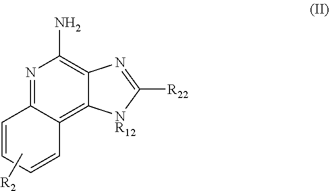
wherein
R₁₂ is selected from the group consisting of straight chain or branched chain alkenyl containing 2 to about 10 carbon atoms and substituted straight chain or branched chain alkenyl containing 2 to about 10 carbon atoms, wherein the substituent is selected from the group consisting of straight chain or branched chain alkyl containing 1 to about 4 carbon atoms and
cycloalkyl containing 3 to about 6 carbon atoms; and cycloalkyl containing 3 to about 6 carbon atoms substituted by straight chain or branched chain alkyl containing 1 to about 4 carbon atoms; and R₂₂ is selected from the group consisting of hydrogen, straight chain or branched chain alkyl containing one to about eight carbon atoms, benzyl, (phenyl)ethyl and phenyl, the benzyl, (phenyl)ethyl or phenyl substituent being optionally substituted on the benzene ring by one or two moieties independently selected from the group consisting of straight chain or branched chain alkyl containing one to about four carbon atoms, straight chain or branched chain alkoxy containing one to about four carbon atoms, and halogen, with the proviso that when the benzene ring is substituted by two such moieties, then the moieties together contain no more than 6 carbon atoms; and each R₂ is independently selected from the group consisting of straight chain or branched chain alkoxy containing one to about four carbon atoms, halogen, and straight chain or branched chain alkyl containing one to about four carbon atoms, and n is an integer from zero to 2, with the proviso that if n is 2, then said R₂ groups together contain no more than 6 carbon atoms; 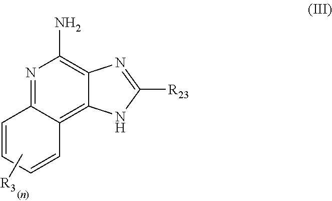
wherein
R₂₃ is selected from the group consisting of hydrogen, straight chain or branched chain alkyl of one to about eight carbon atoms, benzyl, (phenyl)ethyl and phenyl, the benzyl, (phenyl)ethyl or phenyl substituent being optionally substituted on the benzene ring by one or two moieties independently selected from the group consisting of straight chain or branched chain alkyl of one to about four carbon atoms, straight chain or branched chain alkoxy of one to about four carbon atoms, and halogen, with the proviso that when the benzene ring is substituted by two such moieties, then the moieties together—contain no more than 6 carbon atoms; and each R₅ is independently selected from the group consisting of straight chain or branched chain alkoxy of one to about four-carbon atoms, halogen, and 30 straight chain or branched chain alkyl of one to about four carbon atoms, and n is an integer from zero to 2, with the proviso that if n is 2, then said R₃ groups together contain no more than 6 carbon atoms; 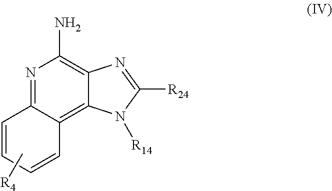
wherein
R₁₄ is —CHR_{A}R_{B} wherein R_{B} is hydrogen or a carbon-carbon bond, with the proviso that when R_{B} is hydrogen R_{A} is alkoxy of one to about four carbon atoms, hydroxyalkoxy of one to about four carbon atoms, 1-alkynyl of two to about ten carbon atoms, tetrahydropyranyl, alkoxyalkyl wherein the alkoxy moiety contains one to about four carbon atoms and the alkyl moiety contains one to about four carbon atoms, 2-, 3-, or 4-pyridyl, and with the further proviso that when R_{B} is a carbon-carbon bond R_{B} and R_{A} together form a tetrahydrofuranyl group optionally substituted with one or more substituents independently selected from the group consisting of hydroxy and hydroxyalkyl of one to about four carbon atoms; R₂₄ is selected from the group consisting of hydrogen, alkyl of one to about four carbon atoms, phenyl, and substituted phenyl wherein the substituent is selected from the group consisting of alkyl of one to about four carbon atoms, alkoxy of one to about four carbon atoms, and halogen; and R₄ is selected from the group consisting of hydrogen, straight chain or branched chain alkoxy containing one to about four carbon atoms, halogen, and straight chain or branched chain alkyl containing one to about four carbon atoms; 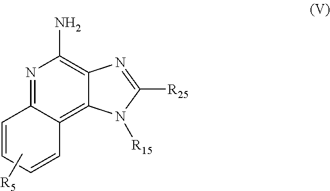
wherein
R₁₅ is selected from the group consisting of: hydrogen; straight chain or branched chain alkyl containing one to about ten carbon atoms and substituted straight chain or branched chain alkyl containing one to about ten carbon atoms, wherein the substituent is selected from the group consisting of cycloalkyl containing three to about six carbon atoms and cycloalkyl containing three to about six carbon atoms substituted by straight chain or branched chain alkyl containing one to about four carbon atoms; straight chain or branched chain alkenyl containing two to about ten carbon atoms and substituted straight chain or branched chain alkenyl containing two to about ten carbon atoms, wherein the substituent is selected from the group consisting of cycloalkyl containing three to about six carbon atoms and cycloalkyl containing three to about six carbon atoms substituted by straight chain or branched chain alkyl containing one to about four carbon atoms; hydroxyalkyl of one to about six carbon atoms; alkoxyalkyl wherein the alkoxy moiety contains one to about four carbon atoms and the alkyl moiety contains one to about six carbon atoms; acyloxyalkyl wherein the acyloxy moiety is alkanoyloxy of two to about four carbon atoms or benzoyloxy, and the alkyl moiety contains one to about six carbon atoms; benzyl; (phenyl)ethyl; and phenyl; said benzyl, (phenyl)ethyl or phenyl substituent being optionally substituted on the benzene ring by one or two moieties independently selected from the group consisting of
alkyl of one to about four carbon atoms, alkoxy of one to about four carbon atoms, and halogen, with the proviso that when said benzene ring is substituted by two of said moieties, then the moieties together contain no more than six carbon atoms;
R₂₅ is 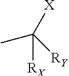
wherein
Rₓ and R_{y} are independently selected from the group consisting of hydrogen, alkyl of one to about four carbon atoms, phenyl, and substituted phenyl wherein the substituent is elected from the group consisting of alkyl of one to about four carbon atoms, alkoxy of one to about four carbon atoms, and halogen; X is selected from the group consisting of alkoxy containing one to about four carbon atoms, alkoxyalkyl wherein the alkoxy moiety contains one to about four carbon atoms and the alkyl moiety contains one to about four carbon atoms, haloalkyl of one to about four carbon atoms, alkylamido wherein the alkyl group contains one to about four carbon atoms, amino, substituted amino wherein the substituent is alkyl or hydroxyalkyl of one to about four carbon atoms, azido, alkylthio of one to about four carbon atoms; and R₅ is selected from the group consisting of hydrogen, straight chain or branched chain alkoxy containing one to about four carbon atoms, halogen, and straight chain or branched chain alkyl containing one to about four carbon atoms; 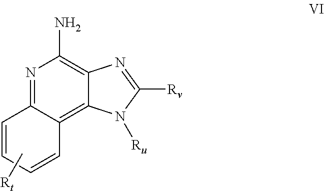
Wherein
Rₜ is selected from the group consisting of hydrogen, straight chain or branched chain alkoxy containing one to about four carbon atoms, halogen, and straight chain or branched chain alkyl containing one to about four carbon atoms;
Rᵤ is 2-methylpropyl or 2-hydroxy-2-methylpropyl; and
Rᵥ is hydrogen, alkyl of one to about six carbon atoms, or alkoxyalkyl wherein the alkoxy moiety contains one to about four carbon atoms and the alkyl moiety contains one to about four carbon atoms.
or a pharmaceutically acceptable salt of any of the foregoing.

6. The composition according to claim 5 wherein the 1H-imidazo[4,5-c]quinolin-4-amine derivative is a compound of formula VI.

7. The composition according to claim 5 wherein Rₜ is hydrogen.

8. The composition according to claim 6 wherein Rᵤ is 2-methylpropyl or 2-hydroxy-2-methylpropyl, and Rᵥ is hydrogen, methyl, or ethoxymethyl.

9. The composition according to claim 7 wherein the compound is selected from the group consisting of
1-(2-methylpropyl)-1H-imidazo[4,5-c]quinolin-4-amine,
1-(2-hydroxy-2-methylpropyl)-1H-imidazo[4,5-c]quinolin-4-amine,
1-(2-hydrozy-2-methylpropyl)-2-methyl-1H-imidazo[4,5-c]quinolin-4-amine, and
1-(2-hydroxy-2-methylpropyl)-2-ethoxymethyl-1H-imidazo[4,5-c]quinoline-4-amine.

10. The vaccine composition according to claim 1 wherein each component is in a form suitable for administration via any of the oral, nasal, topical, pulmonary, intramuscular, subcutaneous or intradermal routes.

11. The vaccine composition according to claim 10 wherein the immunogen component is in a form suitable for administration using a particle mediated gene transfer technique.

12. The vaccine composition according to claim 11 wherein the adjuvant component is in a form suitable for administration using a particle mediated gene transfer technique.

13. A method of inducing an immune response to an antigen, comprising administering either sequentially or simultaneously a nucleic acid encoding an antigen and an imidazo[4,5-c]quinolin-4-amine derivative.

14. A method of inducing an immune response a mammal to an immunogen, comprising the step of administering to said mammal a vaccine composition as claimed in claim1.

15. The method according to claim 14 wherein administration of the adjuvant component takes place on between 1 and 7 occasions, between about 7 days prior to and about 7 days post administration of the immunogen component.

16. The method according to claim 15 wherein administration of the adjuvant component is substantially simultaneous with administration of the immunogen component.

17. The method according to claim 13 wherein the 1 H-imidazo[4,5-c]quinolin-4-amine derivative is administered at a dose of between about 1 mg/kg to 50 mg/kg
